(19) United States
(12) Patent Application Publication
(10) Pub. No.: US 2017/0266006 A1
(43) Pub. Date: Sep. 21, 2017

(54) STENT FOR PERCUTANEOUS VERTEBROPLASTY

(71) Applicant: VARIOMED AG, BALZERS LIECHTENSTEIN (DE)

(72) Inventor: HARALD FISCHER, WEINGARTEN DEUTSCHLAND (DE)

(73) Assignee: VARIOMED AG, BALZERS LIECHTENSTEIN (DE)

(21) Appl. No.: 15/519,080

(22) PCT Filed: Oct. 13, 2015
(86) PCT No.: PCT/EP2015/073633

§ 371 Date: Apr. 13, 2017

(30) Foreign Application Priority Data
Oct. 14, 2014 (DE) 10 2014 114 888.6

(51) Int. Cl. A61F 2/28; A61B 17/88; A61B 17/70

(52) U.S. Cl. CPC A61F 2/2846; A61B 17/7097; A61B 17/8858; A61F 2002/30092

(57) ABSTRACT

A stent for percutaneous vertebroplasty is described having a substantially tubular body that can be transitioned from a compressed state into an expanded state. The wall of the tubular body has a plurality of openings ensuring the expansion both in the longitudinal direction and in the peripheral direction of the stent. The stent has a cross-sectional shape deviating from the circular shape at least in the expanded state.

The present invention relates to a stent for percutaneous vertebroplasty having a substantially tubular body that can be transitioned from a compressed state into an expanded state, wherein the wall of the tubular body has a plurality of openings ensuring the expansion both in the longitudinal direction and in the peripheral direction of the stent.

Such stents are used in percutaneous vertebroplasty to treat vertebral body fractures. In this process, the stents are introduced percutaneously into the vertebral body to be treated in a minimally invasive manner via an application cannula, with the stent being in its compressed state. Once the stent has been positioned within the collapsed vertebral body, which usually takes place under X-ray observation, the stent is transitioned from its compressed state into its expanded state. This is currently typically carried out by a corresponding balloon dilatation. The stent is in this respect positioned within the vertebral body such that, on the expansion of the stent, the collapsed vertebral body is correspondingly expanded until its original vertebral body height is reestablished. At the same time, a cavity for the subsequent injection of bone cement into the vertebral body is created by the expansion of the stent, whereby a final stabilization of the restored vertebral body takes place.

It is problematic with the known stents that in some cases further fractures of the vertebral body can occur due to the forces arising on the expansion of the stent. It is an object of the present invention to provide a stent with which the occurrence of additional fractures on the introduction and expansion of the stent can at least be reduced.

This object is satisfied in accordance with the invention starting from a stent of the initially named kind in that the stent has a cross-sectional shape differing from the circular shape at least in the expanded state.

Known stents for percutaneous vertebroplasty typically have a circular cross-sectional shape both in the compressed state and in the expanded state. The stent is in this respect manufactured, for example, from a tubular body having a small diameter (for example 0.5 to 3 mm) into whose closed body wall openings are cut, for example in slit form or in other forms. On the expansion of the stent, these openings are widened such that a stent having an increased diameter (for example 0.5 to 3 cm) arises. Due to the originally tubular design of the base body with a circular cross-section, the circular cross-section is typically also maintained in this respect in the expanded state, with only the diameter of the circular cross-section being increased. There are furthermore also stents, for example, that comprise mutually woven, welded or otherwise mutually connected wire elements such that here the openings are not formed by corresponding cutting procedures, but rather by free spaces present between individual wire loops. On widening, these free spaces are likewise widened such that the stent can be transitioned from its compressed state into its expanded state. Such stents typically also respectively have a circular cross-section both in the compressed state and in the expanded state since stents have previously always been developed for vessels having a circular cross-section.

It has been found in accordance with the invention that the observed additional fractures are frequently caused on the expansion of the stent in that the contact region between the stent and the vertebral body is substantially restricted to two linear contact regions during the expansion or after the expansion. This follows on from the circular cross-section of the known stents that has the consequence that when the stent is widened, the support surface of the vertebral bodies is respectively limited to a linear support region both at the upper side of the stent and at the lower side of the stent. Since the complete load introduction into the vertebral body thus takes place substantially over these linear support surfaces during the expansion of the stent, additional fractures of the vertebral body frequently arise along these lines on the widening or after the expansion.

Since in accordance with the invention the stent has a cross-sectional shape deviating from the circular shape at least in the expanded state, it is possible to provide a stent in which the support surface of the vertebral bodies is increased on the expansion of the stent or at least after the expansion has taken place. The force acting on the vertebral body on and after the expansion of the stent is thereby distributed over a larger area such that the risk of additional fractures of the vertebral body can be considerably reduced.

The stent advantageously has a circular cross-section in the compressed state that is only changed on the expansion of the stent into an enlarged, non-circular cross-section. This design has the advantage that the stent can be attached to a delivery catheter in the compressed state with a circular cross-section, with the delivery catheter likewise typically having a circular cross-section, whereby, on the one hand, the total diameter of the delivery catheter with applied stent can be kept to a minimum and, on the other hand, no special catheter having a non-circular cross-section is required. The introduction by application needles typically having a circular inner lumen is also simplified with such a configuration. The application needles can furthermore have a minimal inner lumen adapted to the circular cross-section of the compressed stent. It is, however, generally possible that the stent also has a cross-sectional shape deviating from the circular shape in the compressed state. In this case, on the expansion, the general cross-sectional shape of the stent is not changed, but only the size of the cross-sectional surface is increased on the expansion.

In accordance with an advantageous embodiment of the invention, the stent has at least one flattened longitudinal side, in particular two, three, four or more flattened longitudinal sides, at least in the expanded state. It is in particular advantageous that the stent has two flattened longitudinal sides that are arranged opposite one another. The desired increased support surface between the stent and the inner surfaces of the vertebral body is generated by the flattened longitudinal sides on the expansion of the stent. Since the stent is typically supported between two oppositely disposed inner surfaces of the vertebral body, the best force distribution is present in that two flattened longitudinal sides are arranged opposite one another such that the risk of further fractures of the vertebral body on the expansion of the stent can be minimized. The flattened longitudinal sides can in this respect be formed as substantially planar or slightly curved. The curvature may in this respect, however, not be so pronounced that the support surface between the stent and the vertebral body is reduced to a linear support region as with the known stents having a circular cross-section.

If the stent also has a cross-sectional shape deviating from the circular shape in the compressed state, it can have flattened longitudinal sides in the compressed state that correspond to the flattened longitudinal sides in the expanded state.

The stent preferably has a substantially polygonal, rectangular, substantially square or oval cross-sectional shape at least in the expanded state. The cross-section in the compressed state is in this respect preferably circular, but can generally also correspond to the polygonal, rectangular, square or oval cross-section of the expanded state. The polygonal, rectangular, square or oval cross-sectional shape is in this respect not to be understood as mathematically exact. The sides of the polygonal, rectangular or square cross-sectional shape thus, for example, do not have to be exactly of straight-line design, but can rather also be slightly curved so that correspondingly slightly curved support surfaces arise such as was already described in the preceding paragraph. This also applies to the corners of the polygonal, rectangular or square cross-sectional shape that do not have to of exactly right angle design, but can also include an angle slightly different from 90° and/or can also in particular be rounded.

In accordance with a further preferred embodiment of the invention, the stent has a substantially rectangular cross-sectional shape, in particular a substantially square cross-sectional shape, and four flattened longitudinal sides in the expanded state of which a respective two are arranged extending substantially in parallel with one another and opposite one another. The two oppositely disposed longitudinal sides that come into contact with the upper and lower inner surfaces of the vertebral body during expansion thus form surface-optimized, pressure-minimized contact surfaces of the stent. The two longitudinal sides extending perpendicular thereto in contrast form force-optimized erection sides of the stent. The effective erection force or support force of the stent is/are considerably increased with respect to a stent having curved side surfaces due to the flattened configuration, and in particular due to the substantially planar configuration, of these erection sides with a simultaneous alignment perpendicular to the contact surfaces, i.e. in the direction of the support force to be applied during expansion and to be held after expansion. This is particularly advantageous when the stent is configured as a self-expanding stent since here, in comparison with a balloon-expanding stent, the total erection force or at least the substantial erection force has to be generated by the stent itself.

In accordance with a further advantageous embodiment of the invention, the stent has the cross-sectional shape deviating from the circular shape at least over a part region of its longitudinal extent, in particular in a central region. In this respect, the cross-sectional shape differing from the circular shape should in particular be present in that part region of the stent that comes into supporting contact with the vertebral body during the expansion of the stent to form the required support surface between the stent and the vertebral body. The stent can in contrast also in particular have a circular cross-sectional shape at one end or at both ends of the stent as long as the substantial force transmission between the stent and the vertebral body takes place over the part region with the cross-sectional shape differing from the circular form. It is generally also possible that the stent has the cross-sectional shape deviating from the circular shape over its total longitudinal extent.

In accordance with a further advantageous embodiment of the invention, the stent comprises an end tapering in the axial direction at least in the expanded state. The other end of the stent can advantageously likewise be tapered. In this respect, the configuration of the taper, i.e. for example, the corresponding conical angle and the length of the tapered region can be the same at both ends or can differ as required. The tapering ends or the tapering end of the stent can in particular be substantially conical, frustoconical, pyramid-shaped or frusto-pyramidal shaped. It is achieved by the formation of an end tapering in the axial direction that the stent is equipped with an end substantially “closed” at the end face in the expanded state. An unwanted lateral flowing off of the bone cement introduced into the stent after the expansion of the stent is prevented or at least reduced by the end formed as tapering. The tapering can in this respect be configured such that one or both ends of the stent is/are substantially closed or is/are only formed with a relatively small opening after expansion of the stent to prevent or reduce the unwanted flowing out of the bone cement.

In accordance with a further advantageous embodiment of the invention, the other end of the stent substantially has the same cross-sectional shape and/or the same cross-sectional area as the central region of the stent at least in the expanded state. A stent in accordance with the invention can thus have a tapered end that effects a closed or almost closed design of this end of the stent while the other end is provided with an end-face opening that either corresponds to the clearance of the stent in its central region or is only slightly smaller. It is thereby ensured that the delivery catheter that is used for the introduction of the stent and that can also be configured as a balloon catheter, for example, can again be removed in a simple manner through this end face having a larger opening without the position of the stent thereby being changed in an unwanted manner.

In accordance with a further advantageous embodiment of the invention, both ends of the stent substantially have the same cross-sectional shape and/or the same cross-sectional area as the central region of the stent at least in the expanded state. The stent can thus have a uniform width and height or a uniform cross-sectional shape in the expanded state, for example, such that the body of the stent is substantially of parallelepiped shape with open end faces in the expanded state.

The stent can advantageously be configured as a balloon-expandable stent. For example, the stent can comprise stainless steel, titanium or another biocompatible material, in particular metal or plastic. The use of a biodegradable material, in particular of a biodegradable metal or polymer, is also possible. Such a balloon-expandable stent is introduced into a vertebral body using a balloon catheter and is expanded from its compressed state into its expanded state by a corresponding dilatation with a simultaneous widening of the fractured vertebral body. After a restoration of the original vertebral body height has taken place, the balloon catheter is removed and the stent is filled up with bone cement such that the stability of the stent and thus of the vertebral body is strengthened in addition to the stability introduced by the stent.

As already described, it is, however, also possible that the stent is configured as a self-expanding stent. The material of the stent in this respect in particular comprises a memory material, for example a memory alloy (memory metal), in particular Nitinol, or a memory plastic (memory polymer). On the use of a memory material, the stent can be introduced into the vertebral body with the aid of a delivery catheter having a catheter sheath pushed over the stent and can be transitioned from its compressed state into its expanded state by a simple withdrawal of the catheter sheath due to the body temperature after the positioning of the stent. No balloon catheter is thus necessary in this case; with, however, also on the use of a memory material, a balloon catheter generally being able to be used for the additional widening of the stent or for supporting the self-expansion. The stent can in principle completely consist of a corresponding memory material or can only substantially comprise such a memory material. The use of a biodegradable material, in particular of a biodegradable metal or polymer, as the stent material is generally also possible with a self-expanding stent.

Both a balloon-expanding stent and a self-expanding stent can, for example, have an additional biocompatible coating. In this respect, the coating can be configured such that only the material of the tubular body is covered such that the openings ensuring the expansion remain free during the expansion of the stent. It is, however, also possible that the tubular body is covered with a flexible coating, for example a film, that is stretched during the expansion of the stent and that thereby covers over the openings in the wall of the tubular body. A sealed stent, i.e. a stent with a sealed outer wall, can thus be formed in this manner.

Further advantageous embodiments of the invention are set forth in the dependent claims.

The invention will be described in more detail in the following with reference to embodiments and to the drawings; there are shown in these:

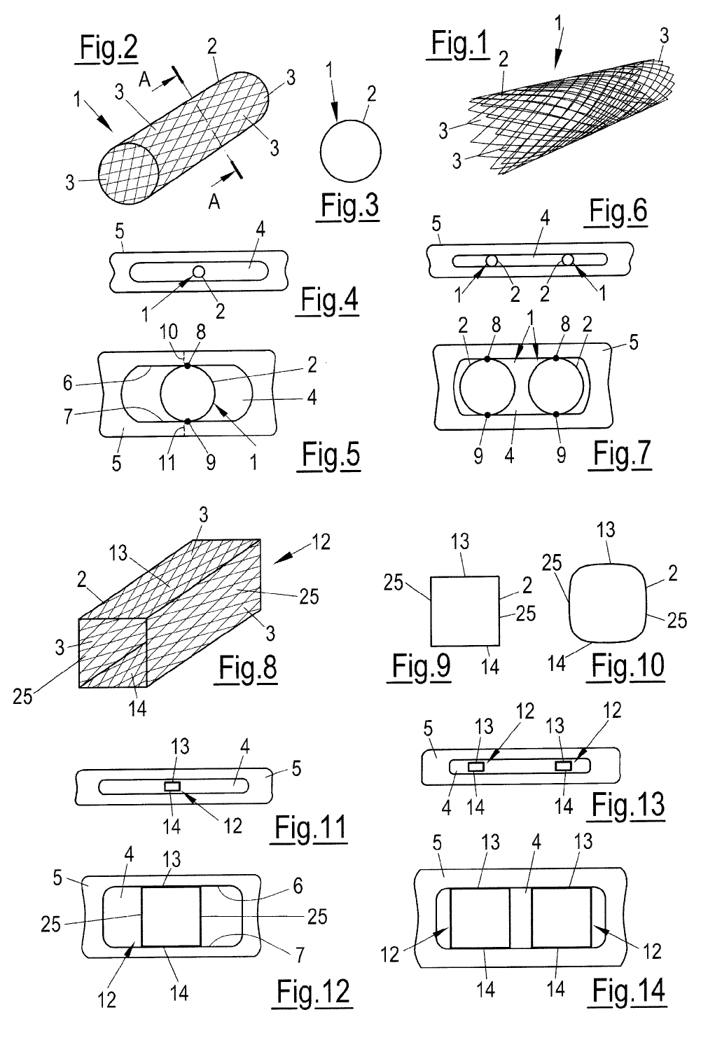

FIG. 1 a perspective representation of a stent for percutaneous vertebroplasty in accordance with the prior art;

FIG. 2 a simplified perspective representation of the stent in accordance with FIG. 1;

FIG. 3 a cross-section along the line A-A of FIG. 2;

FIG. 4 a schematic cross-sectional view of a compressed vertebral body with an inserted stent in accordance with FIG. 1 in the compressed state;

FIG. 5 a vertebral body in accordance with FIG. 4 with an expanded stent;

FIG. 6 a further compressed vertebral body with two inserted stents in accordance with FIG. 1 in the compressed state;

FIG. 7 the vertebral body in accordance with claim 6 with expanded stents;

FIG. 8 a schematic perspective representation of a stent in accordance with the invention with a square cross-section;

FIG. 9 a cross-section through the stent in accordance with FIG. 8;

FIG. 10 a slightly modified cross-sectional shape;

FIG. 11 a schematic cross-sectional view of a compressed vertebral body with an inserted stent in accordance with FIG. 8 in the compressed state;

FIG. 12 the vertebral body in accordance with FIG. 11 with an expanded stent;

FIG. 13 a schematic view of a compressed vertebral body with two inserted stents in accordance with FIG. 8 in the compressed state;

FIG. 14 the vertebral body in accordance with FIG. 13 in the expanded state;

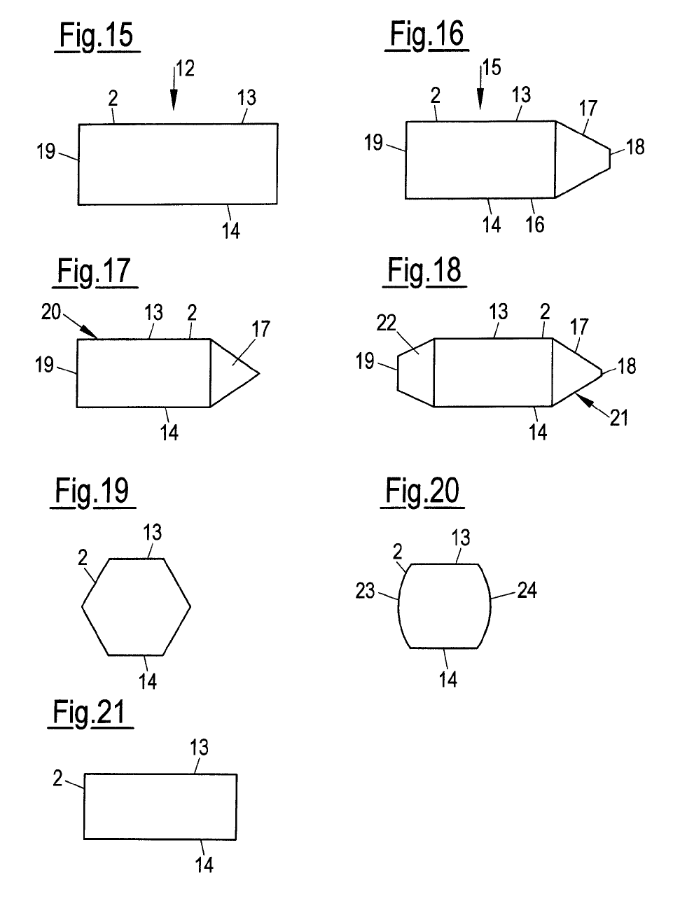

FIG. 15 a schematic side view of the stent in accordance with FIG. 8;

FIGS. 16 to 18 further schematic side views of stents in accordance with the invention; and

FIGS. 19 to 21 schematic representations of different cross-sectional shapes of a stent in accordance with the invention.

In all the embodiments, the same, similar or mutually corresponding elements are marked by the same reference numerals.

FIG. 1 shows a stent 1 having a tubular body 2 that has a mesh structure such that a plurality of openings 3 are formed in the wall of the body 2 that repeat both in the longitudinal direction and in the peripheral direction of the stent 1. FIG. 2 shows the same stent 1 in a simplified perspective representation from which it can in particular clearly be recognized that the stent 1 has a circular cross-section. The circular cross-section of the stent 1 is in this respect identical over the total length of the stent 1 such that the body 2 forms a cylinder jacket having openings 3. The openings 3 are in this respect of diamond shape and are created in that the stent 1 is transitioned from a compressed state, not shown, having a clearly reduced diameter, into the expanded state shown in FIG. 1 and FIG. 2, with the openings 3 being widened to enable the expansion. In the compressed state of the stent 1, the openings 3 can, for example, be formed as slit-like openings that extend in parallel next to one another in the axial direction, with slit-shaped openings respectively disposed next to one another in the peripheral direction being arranged alternately offset with one another.

The circular cross-section of the stent 1 is shown again in a simplified form in FIG. 3.

The stent in accordance with FIGS. 1 to 3 is a stent for percutaneous vertebroplasty known from the prior art. In accordance with FIG. 4, such a stent is inserted in the shown compressed state, i.e. with a reduced diameter, into the vertebral body cavity 4 of a collapsed vertebral body 5. The insertion in this respect takes place in a known manner by means of a delivery catheter.

The stent 1 is widened, for example via a balloon catheter, into its expanded position shown in FIG. 5 after a successful placement. Due to the circular cross-section of the stent 1, it only comes into contact with the inner sides 6, 7 of the vertebral body 5 via linear support points 8, 9 via which the total erection force is introduced into the vertebral body 5 on a further expansion of the stent. An additional fracture point can therefore arise in the region of the linear support points 8, 9 during the expansion, such as are indicated by dashed lines 10, 11.

This problem also occurs when, as shown in FIGS. 6 and 7, two stents 1 are inserted into the vertebral body cavity 4 and are subsequently expanded in the usual manner for widening the vertebral body 5. Both stents 1 are here also only in contact with the inner sides 6, 7 of the vertebral body 5 via linear support points 8, 9 such that the material of the vertebral body 5 is extremely strained in these regions.

FIG. 8 shows a stent 12 in accordance with the invention in a likewise simplified perspective representation that, unlike the previously described stent 1, has a square cross-section such as is shown schematically in FIG. 9. Unlike the stent 1, the stent 12 in accordance with the invention has a cross-sectional shape deviating from the circular shape and in particular has two oppositely disposed flattened longitudinal sides 13, 14 that form enlarged support surfaces such as will be explained in more detail in the following. The two other oppositely disposed longitudinal sides 25 of the stent 12 are likewise correspondingly flattened. As can be recognized from FIG. 10, the cross-section of the stent 12 does not have to be exactly mathematically square in this respect, but the side surfaces can rather be slightly arcuate and the corners can be rounded as long as the flattened longitudinal sides 13, 14 form support surfaces that are not only in linear contact with the inner sides 6, 7 of the vertebral body 5, but rather form areal contact regions therewith.

This can be recognized from FIGS. 11 and 12. The stent 12 in accordance with the invention is inserted into the vertebral body cavity 4 of the collapsed vertebral body 5 in its compressed state in FIG. 11. If the stent 12 is transitioned from its compressed state into the expanded state shown in FIG. 12, the two flattened longitudinal sides 13, 14 are pressed areally toward the inner sides 6, 7 of the vertebral body 5 such that the force transferred from the stent 12 to the vertebral body on the expansion is distributed over a larger surface and the vertebral body 5 is thus not strained in a linear manner as with conventional stents. The risk that an additional fracture of the vertebral body 5 occurs on the expansion of the stent 12 and on the widening of the vertebral body 5 is thereby minimized. Furthermore, it is achieved by the likewise flattened longitudinal sides 25 that simultaneously extend perpendicular to the support surfaces (and thus in the direction of the erection movement of the vertebral body 5) that the erection force that can be generated by the stent 12 is considerably increased with respect to the erection force of the stent 1.

If a stent is used that has a circular cross-section in the compressed state, it is advantageous if the cross-sectional shape changes at a relatively early point during the expansion from the circular shape into a cross-sectional shape differing from the circular shape, in particular having oppositely disposed flattened longitudinal sides. It is thereby ensured that the force transmitted to the vertebral body during the expansion is distributed over a larger surface during the total expansion process or at least during a large part of the expansion process. It is, however, generally also conceivable that the cross-sectional shape of the stent only changes into a shape deviating from the circular shape briefly before or on the reaching of the expanded state such that substantially only the final support of the widened vertebral body takes place over increased surfaces. These possible embodiments are also applicable to the further embodiments described in the following. Such a stent in accordance with the invention can thus be configured in accordance with FIG. 4 in the compressed state and in accordance with FIGS. 8 to 10 and 12 in the expanded state.

On a use of two stents 12 in accordance with the invention, such as is shown schematically in FIGS. 13 and 14, the total support surface, and thus the total surface for the force transmission from the stents 12 to the vertebral body 5, is further increased such that the risk of an additional fracture of the vertebral body 5 can be further reduced. The stents 12 can here, deviating from the representation in accordance with FIG. 13, also have a circular cross-section in the compressed state. The stents 12 can thus be configured in accordance with FIG. 6 in the compressed state and in accordance with FIGS. 8 to 10 and 14 in the expanded state.

The expansion of the stent 12 can in this respect take place via balloon dilatation. It is, however, also possible that the stent 12 is a self-expanding stent that is automatically transitioned after placement by the body temperature from its compressed state into its expanded state due to its formation from a memory material such as Nitinol. A cross-section circular in the compressed state and a cross-section at least regionally rectangular or square in the expanded state can in particular be imparted to the self-expanding stent on its manufacture.

FIGS. 15 to 18 show side views of differently configured stents in accordance with the invention in a schematic representation. FIG. 15 in this respect shows a side view of the stent 12 of FIG. 8, with, as also in the following Figures, only the outline of the side view being shown. Since the stent 12 in accordance with FIG. 8 has a parallelepiped-shaped body, the outline of the side view only forms the rectangle shown in FIG. 15. Both the cross-sectional shape and the cross-sectional size are thus identical over the total length of the stent in this stent.

The outline of a side view of a modified stent 15 is shown in FIG. 16. In this stent 15, a main part 16 is configured in accordance with the stent 12, i.e. with a square cross-section. A conically tapering end 17 adjoins this main part 15 and opens in an end-face opening 18 of the stent 15. The oppositely disposed end-face opening 19 of the stent 15 in contrast has a clearance that corresponds to the inner lumen of the stent 15 in its main part 16.

With the stent 15, for example, the delivery instruments for introducing and placing the stent 15 can be guided through the large end-face opening 19 such that the delivery catheter can be withdrawn from the stent 15 without problems after the placement of the stent. Bone cement can furthermore be injected into the interior of the stent 15 in a simple manner via the larger end-face opening 19 to improve the stability of the stent 15 and thus of the expanded vertebral body 5. It is prevented by the tapering end 17 of the stent having the reduced end-face opening 18 that the bone cement introduced into the interior of the stent 15 can exit the distal side of the stent 15 again. The tapering end 17 can in this respect, for example, be of frusto-pyramidal shape or of frustoconical shape, i.e. can be formed with a square or rectangular cross-section, with a polygonal cross-section or with a circular cross-section. This also applies to the tapering sections of the following stents described in the following.

The stent 20 shown in a side view in FIG. 17 only differs from the stent 15 in accordance with FIG. 16 in that the tapering end 17 does not form an open end of the stent 20, but rather a closed end. An outflow of the bone cement injected into the stent 20 is thereby completely or at least largely prevented.

It can be recognized from FIG. 18 that a stent 21 in accordance with the invention can have two tapering ends 17, 22. As is shown in FIG. 18, the two tapering ends 17, 22 can form different end-face openings 18, 19 of the stent and can also have different cone angles. The two tapering ends 17, 22 can generally also be identical.

If the proximal end of the stent is formed as tapering, the stent can, when necessary, for example on an incorrect positioning, be withdrawn into the delivery catheter again and can subsequently be repositioned.

It is common to all the stents shown that they have a cross-section deviating from the circular shape at least in the expanded state and, for example, as shown in FIGS. 9 and 10, have a square cross-section. Deviating from the square cross-section, stents in accordance with the invention can, for example, also have cross-sectional shapes such as are shown in FIGS. 19 to 21. A hexagonal cross-section is shown in FIG. 19, for example, whereby in turn two oppositely disposed flattened longitudinal sides 13, 14 are implemented. The same also applies to the cross-section shape in FIG. 20 in which the oppositely disposed flattened longitudinal sides 13, 14 are connected to one another by curved side surfaces 23, 24.

It is shown in FIG. 21 that the cross-section of a stent in accordance with the invention can also be configured as an elongated rectangle so that, for example, as shown in FIG. 14, two stents 12 arranged next to one another can be replaced with a simple stent in accordance with the invention having a larger width.

### REFERENCE NUMERAL LIST

1 stent
2 body
3 openings
4 vertebral body cavity
5 vertebral body
6 inner side
7 inner side
8 linear support point
9 linear support point
10 fracture point
11 fracture point
12 stent
13 flattened longitudinal side
14 flattened longitudinal side
15 stent
16 main part
17 tapering end
18 end-face opening
19 end-face opening
20 stent
21 stent
22 tapering end
23 curved side surface
24 curved side surface
25 flattened longitudinal sides

1. A stent for percutaneous vertebroplasty having a substantially tubular body that can be transitioned from a compressed state into an expanded state, and having a central region wherein a wall of the tubular body has a plurality of openings ensuring the expansion both in the longitudinal direction and in the peripheral direction of the stent,
wherein the stent has a cross-sectional shape deviating from the circular shape at least in the expanded state.

2. The stent in accordance with claim 1,
wherein the stent has a circular cross-section in the compressed state.

3. The stent in accordance with claim 1 or claim 2,
wherein
the stent has at least one flattened longitudinal side, at least in the expanded state.

4. The stent in accordance with claim 3,
wherein the stent has two, three, four or more flattened longitudinal sides, at least in the expanded state.

5. The stent in accordance with claim 3,
wherein
the stent has two flattened longitudinal sides that are arranged disposed opposite one another.

6. The stent in accordance with claim 1,
wherein the stent has a substantially polygonal, rectangular, substantially square or oval cross-sectional shape at least in the expanded state.

7. The stent in accordance with claim 1,
wherein
the stent has a substantially rectangular cross-sectional shape in the expanded state.

8. The stent in accordance with claim 7,
wherein the stent having the substantially square cross-sectional shape has four flattened longitudinal sides in the expanded state of which two respective longitudinal sides are arranged extending in parallel with one another and disposed opposite one another.

9. The stent in accordance with claim 1,
wherein the stent has the cross-sectional shape deviating from the circular shape at least over a part region of its longitudinal extent.

10. The stent in accordance with claim 9,
wherein the stent has the cross-sectional shape deviating from the circular shape at least in the central region of the stent.

11. The stent in accordance with claim 1, wherein the stent has the cross-sectional shape deviating from the circular shape over its total longitudinal extent.

12. The stent in accordance with claim 1,
wherein the stent comprises a tapering end tapering in the axial direction at least in the expanded state.

13. The stent in accordance with claim 12,
wherein the other end of the stent is likewise formed as tapering.

14. The stent in accordance with claim 12,
wherein the tapering end of the stent is substantially conical, frustoconical, pyramid-shaped or frusto-pyramidal shaped.

15. The stent in accordance with claim 12,
wherein the other end of the stent substantially has the same cross-sectional shape and/or the same cross-sectional area as the central region of the stent at least in the expanded state.

16. The stent in accordance with claim 1,
wherein both ends of the stent substantially have the same-cross-sectional shape and/or the same cross-sectional area as the central region of the stent at least in the expanded state.

17. The stent in accordance with claim 1, wherein the stent is configured as a balloon-expandable stent.

18. The stent in accordance with claim 1, wherein the stent is configured as a self-expanding stent.

19. The stent in accordance with claim 18, wherein a material of the stent comprises a memory material.

20. The stent in accordance with claim 19, wherein the stent substantially or completely consists of a memory material.
